(19) United States
(12) Patent Application Publication
(10) Pub. No.: US 2018/0339216 A1
(43) Pub. Date: Nov. 29, 2018

(54) FOAM ROLLER AND ROLLER INSERT MODULE FORMUSICAL THERAPY AND METHOD OF USE

(71) Applicant: Mario Gencarelli, Cedar Knolls, NJ (US)

(72) Inventor: Mario Gencarelli, Cedar Knolls, NJ (US)

(21) Appl. No.: 15/990,659

(22) Filed: May 27, 2018

Related U.S. Application Data
(60) Provisional application No. 62/511,859, filed on May 26, 2017.

(51) Int. Cl. A63B 71/06; A61M 21/02; A61H 7/00; A63B 21/00; A63B 24/00

(52) U.S. Cl. CPC A63B 71/0622; A61M 21/02; A61M 2021/0027; A63B 21/0004; A63B 24/0075; A61H 7/003

(57) ABSTRACT

An improved foam roller having a sound card and one or more speakers included therewith so that the user may perform his/her exercises to music for greater motivation, adherence and compliance to an exercise regimen. Preferred embodiments would include Bluetooth® connectivity and/or a USB port for downloading one's own preferred playlist. A timer with repetitions indicator would also be preferred. This device could be installed in a new or existing, cylindrically-shaped foam roller. A method for exercising with this improved foam roller is also disclosed herein.

### CROSS-REFERENCE TO RELATED APPLICATION

This is a perfection of U.S. Provisional Application Ser. No. 62/511,859, filed on May 26, 2017, the disclosure of which is fully incorporated by reference herein.

### BACKGROUND OF THE INVENTION

A main concern during rehabilitation and treatment of an athlete is the athlete's adherence and compliance during treatments for soft tissue injuries, muscle stiffness, delayed onset muscle soreness (DOMS) and structural deformities. In most clinical settings, the foam roller or self-myofascial release (SMR) has been integrated into the foundational routine for recovery.

Curran et al¹ states that over the past decade SMR has become a common technique to treat myofascial restrictions and restore normal soft-tissue extensibility. SMR is increasingly used as a method to supplement traditional methods of treating soft-tissue injuries. Prior to this invention, however, music has never been coupled to SMR in any known manner.

One strategy for increasing duration and motivation in patients receiving care is musical therapy. When music gets incorporated into gait and lower limb physical rehabilitation, for example, Weller et al² found that music and rhythm provide motivation, anticipation, time reference, direct attention, increased engagement and help with stimulating long-term memory.

The focus of this invention was to research the benefits of increased foam rolling on the body physiologically, especially when combined with music therapy, to aid in the compliance and adherence during sessions. Accordingly, this invention focused on designing and creating a foam roller that combines with some sort of musical therapy such as a radio or other musical playback device.

### Foam Roller Overview

Foam rollers are cylinders made of varying densities and textures of foam wrapped about a plastic inner foundation that gives the roller its firm internal, cylindrical base. In one study, Couture et al³ found that these tools, i.e. foam rollers, utilize the same mechanism of treatment as traditional myofascial release, but with pressure being applied by the individual externally or using his/her own body weight. During a foam rolling regimen, the athlete will focus on specific musculature to release muscle tension and soreness while incorporating supporting musculature to aid in the foam roller's benefits and efficiencies. Foam rolling has been perceived to increase range of motion (ROM) and muscle extensibility while also decreasing stiffness and muscle soreness. If the duration of exercising each body part was increased, this could lead to improved function during a given activity. Foam rolling per se is a form of self-myofascial release that can be integrated into a rehabilitation protocol during the warm up phase of treatment to: (i) allow the muscles to actively warm up; (ii) increase muscle extensibility; (iii) increase range of motion and (iv) decrease muscle soreness.

In a recent study, Vaughan et al⁴ mentioned that foam rollers are being more widely used in sport and rehabilitation settings to: achieve changes in muscle tone; restore tissue extensibility, and increase range of motion. Their research showed that athletes who used a foam roller as part of their warm-up routine perceived less muscle fatigue, even though there was no difference in athletic performance between groups.

### Foam Rolling Duration

While researching the most efficient duration for utilizing a foam roller/self-myofascial release, Couture et al³ stated that recommended durations of application have varied, from between about 60 to 90 seconds to up to about five minutes . . . or until a release is felt. Total duration should include each individual muscle being foam rolled for a set time depending on the athlete's soreness and soft tissue injury. Physiologically when the muscles are being foam rolled, the main focus is on the fascia covering the muscle, i.e., that is a tough sheet of connective tissue which envelops and binds together the soft tissue of the body. With an increased duration and utilization of the SMR, one could conceivably increase the break up (or release) of fibrosis adhesions between layers of the myofascial system to: aid in normal biomechanics, increase ROM, correct muscular imbalances and increase soft tissue extensibility.

In their study, Curran et al′ noticed that SMR is a common therapy used for increasing soft-tissue extensibility and decreasing muscle soreness.

In yet another study, Couture et al³ found a correlation with “foam rolling”, i.e., utilizing a PVC pipe covered in foam, on the quadriceps for one to two minutes. They saw a 12.7% increase in quadriceps ROM two minutes after such foam rolling ended. A trend noticed in most publications is that an average foam-rolling time ranging from one to two minutes on a particular muscle group can lead to potential physiological benefits. Similar evidence was present by Murray et al⁵. They found a greater effect with a greater force yielding meaningful improvements after only about one to two minutes of treatment.

As we strive to find a correlation with the efficiency and benefits of increased foam roller use durations, other studies focused what effect changing the amount of repetitions had. Couture et al³, for example, found that increasing the amount of one minute session repetitions saw a 12.7% increase in ROM—particularly for two minutes after two, one minute bouts of foam rolling. They also observed a 16% ROM increase after five, one minute bouts of foam rolling. Integrating multiple repetitions should be considered as a way to further increase the physiological benefits of foam rolling while we work on finding ways to increase an athlete's adherence and compliance to such regimens.

### Foam Rolling Benefits

Foam rolling/self-myofascial release is a vital method in rehabilitation treatment, especially during the warm up to an activity. Incorporating foam rolling allows the muscles to actively warm up by increasing blood flow to specific parts of the body, increasing muscle extensibility and ROM, and decreasing muscle soreness. Vaughn et al⁴ stated that foam rollers are becoming more widely used in sport and rehabilitation settings to: achieve changes in muscle tone, restore tissue extensibility and increase ROM.

In their study, Healey et al⁶ found that, similar to massage, foam rolling before a workout helps restore muscle length—tension relationships and allow for a better warm-up. Regarding the increase of blood flow from foam rolling before an activity, Mohr et al⁷ claimed that SMR usage improves ROM through an autogenic inhibition whereas massage increases blood flow and reduces adhesions and scar tissue.

### Music Therapy

Weller et al² recognized that music per se (i.e., NOT in association with any SMR prior to the present invention): (a) increases level of engagement, (b) enhances physical functioning and psychosocial functioning, (c) helps patients remember stored movement programs in long-term memory, (d) decreases perceived subjective exertion and muscle lactate during physical training and (e) produces a state for relaxation. Music is a tool used many individuals in exercise workouts and daily activities. As part of this investigation, applicant asked: will the addition of music increase one's adherence and compliance during foam rolling/SMR while further encouraging the athlete to use his/her foam roller more—i.e., for longer times and/or greater numbers of repetitions. Once more, in a non-SMR context, Alter et al⁸ suggested that tempo-pace synchronous music can increase exercise endurance, augment its intensity and lead to extended durations of exercise. Would the same apply to foam rolling/SMR?

When utilizing a foam roller, many individuals do not understand or fully appreciate the physiological benefits associated with its use. That leads to a decreased adherence, i.e., the number of repetitions and time spent on specific muscle groups. With the addition of music to the “mix”, especially from a playlist provided by the exercising individual, compliance with using the foam roller for a recommended time (duration and/or number of rep's) may increase. The earlier Alter et al⁸ study found that when coupled with individual preferences, music alone may enhance motivation, improve affect, induce distraction and attenuate a patient's perceptions of effort during exercise. Imagine what using music in an SMR context might further accomplish . . . .

### Music Therapy on Duration of Activity:

While researching evidence-based medicine, a correlation was found that music may change one's perception of effort during exercise, enhancing motivation, and reducing distractions. That, in turn, could lead to an increase of duration and compliance during foam roller utilization. In a study named MAINTAIN, for “the Music Activity Intervention for Adherence Improvement Through Neurological” entertainment, another musical research question was explored. Answers from the latter MAINTAIN study were mentioned by Alter et al⁸. They opined that that the feasibility and efficacy of implementing a tempo-pace, synchronized personalized audio-playlist device may improve adherence to physical activity among patients participating in a structured exercise program. They also concluded that differences in weekly physical activity volumes were greatest among those receiving rhythmic auditory stimuli embedded into their audio-music playlists.

Music has the ability to motivate people to become active and sustain activity. Applicant believes that this could this translate into increased repetitions, proper duration and weekly utilization during foam rolling. Weller et al² noted that visual and auditory feedback may have produced an increased motivation for frequent repetitions of identical movements thereby increasing rehabilitation potential. Will that safe effect be observed in a purer foam roller/SMR context?

To date, there have been no known studies showing music alone as a catalyst for increasing motivation, increasing engagement and decreasing perceived exertion during a foam roller rehabilitation exercise regimen. Alter et al⁸ merely shared results which indicated that motivational synchronous music may elicit an ergogenic effect and enhance in-task affects during an exhaustive endurance task. Meanwhile, Weller et al² noted that the employment of music and rhythm may provide motivation, anticipation, time reference, direct attention, and an increased engagement to/stimulation of long-term memory.

### Other Relevant Art

No references were found teaching or suggesting the use of any sound-related additions to (or incorporations into) new or existing foam rollers, internal or otherwise. There were multiple instances of heat and/or vibrational mechanisms added to such devices (or an external cover thereto). In chronological order, they include: Egan et al U.S. Published Application No. 2016/0279018, Phillips U.S. Pat. No. 9,005,146, Heller U.S. Published Application No. 2015/0065924, Diecidue U.S. Published Application No. 2014/0371639, Bennett U.S. Published Application No. 2013/0231594, Abdur-Raoof U.S. Published Application No. 2013/0178766, Brinson U.S. Published Application No. 2009/0176635 and Barrows U.S. Pat. No. 7,137,926 to name a few.

There were also several known references to the inclusion of Bluetooth® with speakers to a common sleeping pillow. U.S. Pat. No. 4,782,533 and Korean Patent No. 20160123109 are representative of this art. It should be noted that Applicant is not claiming to have invented sound cards or speaker systems per se. However, through the addition of a sound card to a new, or previously existing, foam roller, Applicant's method of inputting preferred music through such sound systems should enhance an athlete's adherence to his/her use of a foam roller for pre-activity warm ups and/or massage-related muscle therapy sessions. Next-gen variations of Applicant's device may simplify usage even further by graphically depicting (on the foam roller proper) a visual body indicator AND timer so that the user may touch the illustrated body part on the roller and start the amount of time recommended to fully and fairly exercise that same body part (in one or more repetitions). An audible alarm would sound at the end of the one SMR session (or after every timed repetition) for the body part in question.

Alternately, the foam roller of the future can be provided with a video display screen either as part of an end cap (see, FIG. 3C) or mid-cylindrical body. When the user touches a particular body part on that video display to exercise, a video for exercising that particular part would be displayed BEFORE the timer commences for exercising that body part per the previously displayed video.

### BRIEF SUMMARY OF THE INVENTION

This invention addresses an improved foam roller having a sound card and one or more speakers included therewith so that the user may perform his/her exercises to music for greater motivation, adherence and compliance to an exercise regimen. Preferred embodiments would include Bluetooth® connectivity and/or a USB port for downloading one's own preferred playlist. A timer with repetitions indicator would also be preferred. This device could be installed in a new or existing, cylindrically-shaped foam roller. A method for exercising with this improved foam roller is also disclosed herein.

### BRIEF SUMMARY OF THE DRAWINGS

Further features, objectives and advantages of this invention will be made clearer from the following detailed description made with reference to Applicant's accompanying drawings in which:

FIG. 1 is a top perspective view of one embodiment of foam roller according to this invention;

FIG. 2 is a top perspective view of the lateral (power) end of the foam roller from FIG. 1;

FIG. 3A is a top perspective view of the opposite end of the foam roller from FIGS. 1 and 2 with its full speaker shown;

FIG. 3B is a top perspective view of a first alternate end cap showing a body part display map, timer, USB port and smaller side speaker;

FIG. 3C is a top perspective view of a second alternate end cap showing a video screen display surrounded by a smaller, circumferential side speaker;

FIG. 4 is a top perspective, close up view of one type of sound speaker, from an end of the foam roller from FIGS. 1 through 3A; and

FIG. 5 is a top perspective view of the sound card components for installing inside a new, or an existing, foam roller per the present invention.

### DESCRIPTION OF PREFERRED EMBODIMENTS

Recommendations—Overall, foam rolling/SMR is a known tool utilized by many healthcare professions to aid their athletes and/or patients recovering from specific soft tissue injuries. Foam rollers are also used in the warm-up phase of an activity to aid in soft tissue extensibility, muscle extensibility, increasing blood flow to muscles and/or to reduce fatigue and DOMS. Utilizing this tool can be mildly painful due to the depth of massaging factors from the roller itself. But the benefits from use outweigh the minor pain(s) observed from typical roller utilization. By combining foam roller use with music, Applicant hopes to motivate an individual to better use his/her foam roller—for the recommended time frame and/or increased number of repetitions thus further reducing fatigue potential and the delayed onset of muscle soreness and pain.

In their study, Weller et al² noted that music (in a NON-SMR context) had the potential to: increase level of engagement, decrease perceived subjective exertion and muscle lactate during physical training, and produce a state for relaxation. This invention takes the knowledge of music and purposefully combines same with the growing knowledge of benefits from foam roller exercising/treatment sessions to potentially aid in further increasing the physiological benefits, adherence and compliance observed.

### Structure of Project Design:

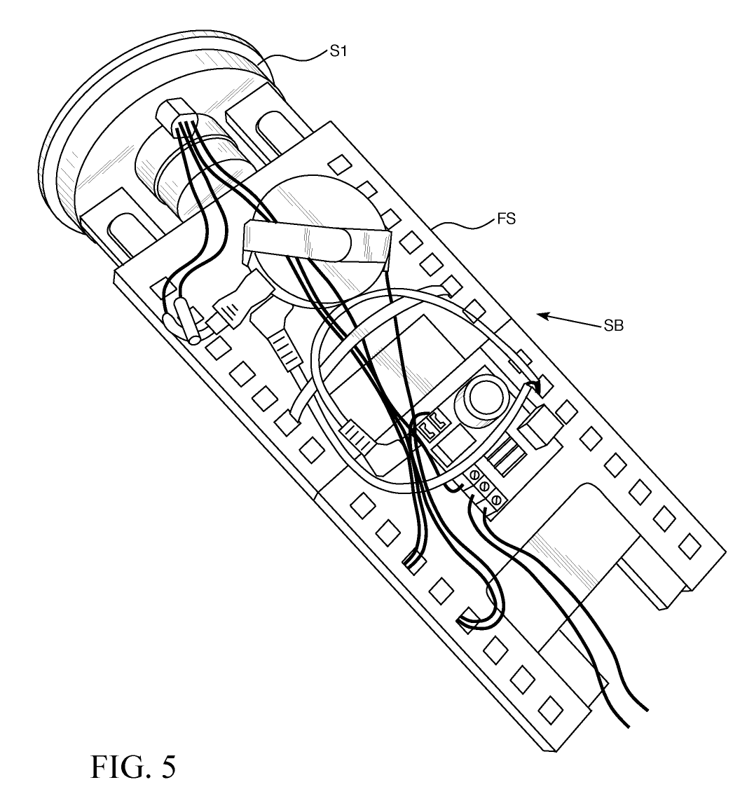

Applicant's research was guided toward utilizing a foam roller, in combination with music, to innovate a foam roller design that incorporates harmonizing speakers and Bluetooth® capabilities into the foam roller exercising regimen. This included structuring the coding for Bluetooth capabilities while piecing together components that would allow for music to be played from within a DURABLE foam roller exercising device knowing the degrees to which foam rollers (without musical components) are subjected to physical abuses inside a typical exercise room and/or training facility. This invention does not merely add a radio to the interior of a cylindrical foam roller body. The components of the sound card shown in FIG. 5 must withstand the rigors of being tossed about, from one user to another, in an athletic training room, and/or repeatedly rolled over by the full body weight of an individual using that roller to perform warm up and/or treatment exercises therewith.

### Marketing Strategy:

Foam rollers are a continuously expanding tool for therapeutic and functional benefits in secondary schools, university/colleges, professional facilities, therapeutic facilities, fitness centers and hospitals. The foam roller can be utilized by individuals of any age to help contribute to that user's increased ROM and blood flow, decreased muscle soreness and/or to otherwise help prepare for his/her upcoming (warm up) activities. This invention improves upon the basic foam roller by integrating Bluetooth-synchronized speakers that can relate to increased adherence and workout compliance by athletes and patients alike.

### Strengths:

A truly innovative improvement to the foam roller design that can translate into increased duration, adherence, and physiological benefits from an extended, yet comfortable music-stimulated foam roller session or sessions.
The market range for the foam roller product attracts individuals of all ages.
Attracts a large array of the health care professions ranging from therapeutic settings to hospitals, fitness centers, high schools, and professional facilities.
Easy to use, Bluetooth® capabilities that will produce an efficient sound quality under otherwise less than ideal (potentially wet and sweaty) conditions.
A more functional Bluetooth® capability will allow each foam roller user to customize his/her music of choice to be played there through.
An internalized music source will remove the possibility of any/all interference from external music headphone wires.
Combines foam rollers and audio systems at a reasonable, reduced cost.

### Opportunity:

Distributed to national and international markets regarding therapeutic settings to hospitals, fitness centers, high schools, and professional facilities.
Large market circulation which will translate into availability for still further future advancement(s).

### Current Prototype

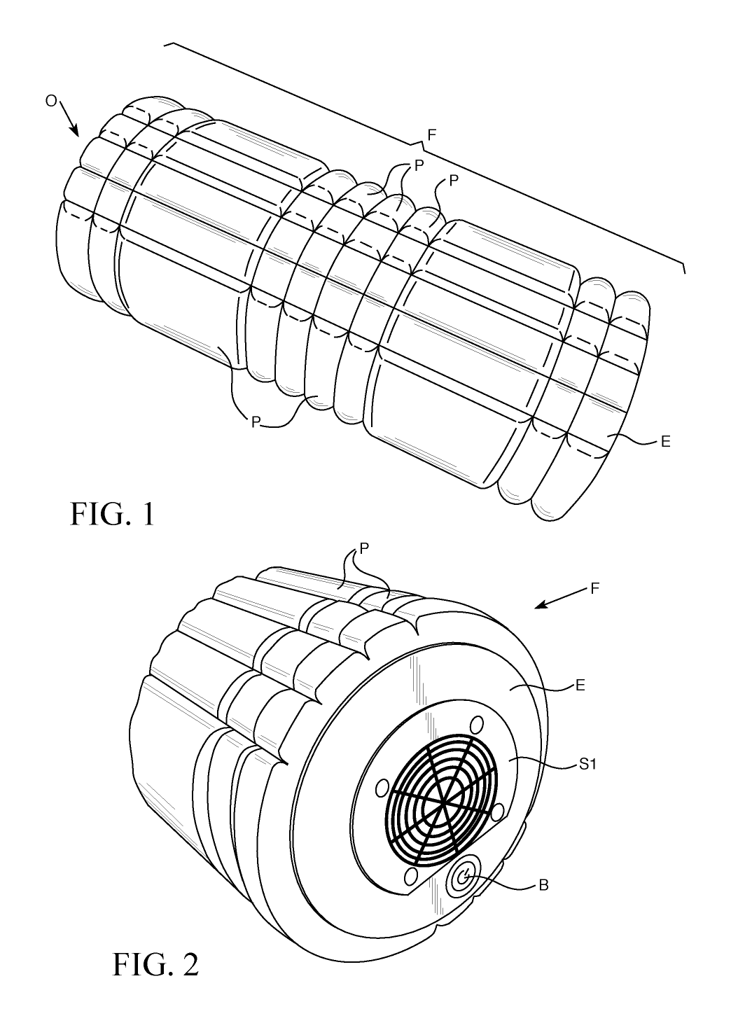

Referring to accompanying FIGS. 1, 2, 3A, 4 and 5, there is shown a first model of foam roller F that is columnar in shape with a circularly-shaped cross-section (as opposed to slightly oval, square or triangular shaped alternative). Foam roller F has a plurality of padded regions P, in varying shapes and sizes, about its exterior skin. Though not shown, it may be possible to encase the main cylindrical body (excluding its speaker end caps) of foam roller F in a towel or other sweat absorbing cover for greater hygiene between users. That cover may be washed and replaced or made to be disposable.

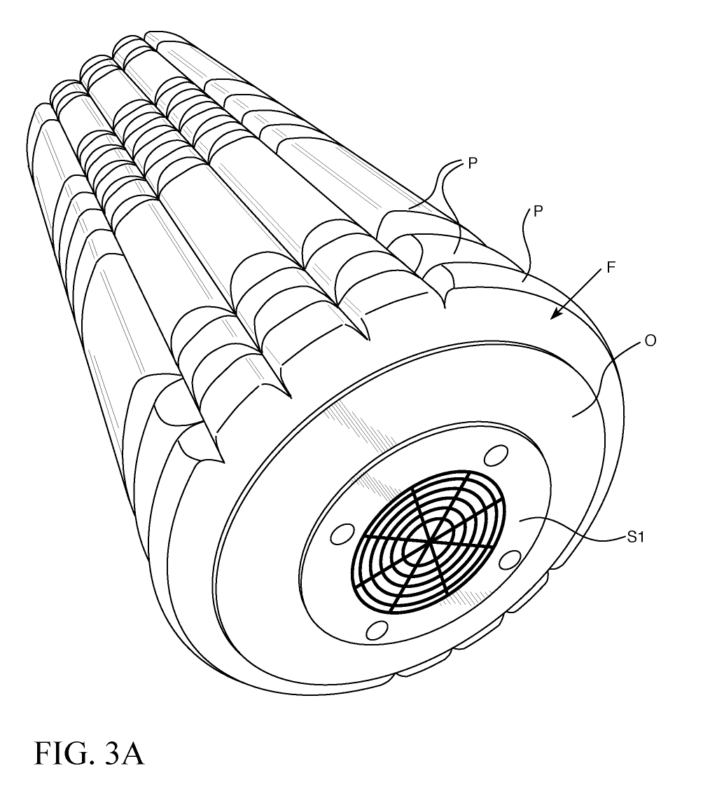

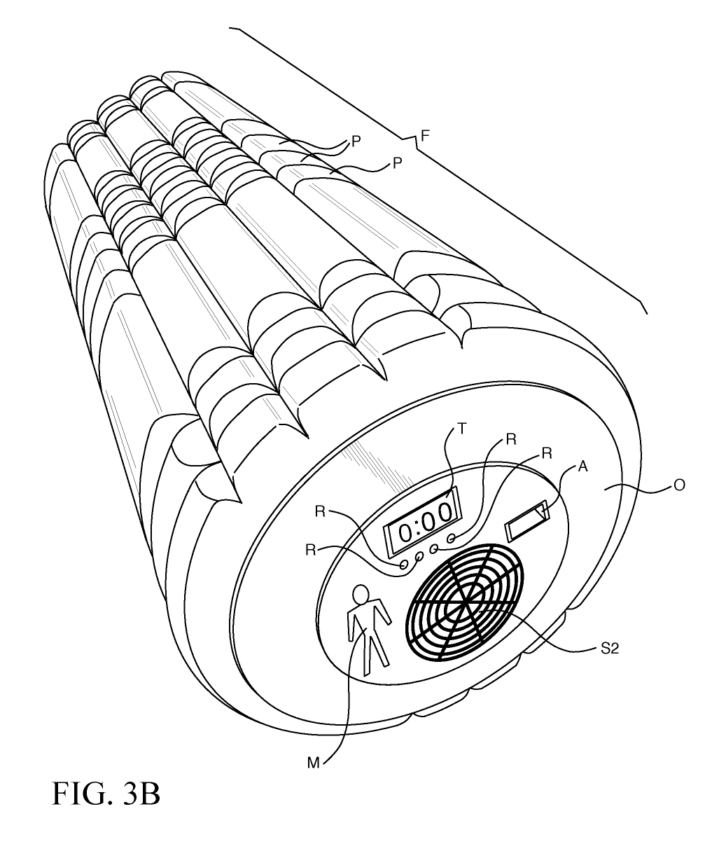

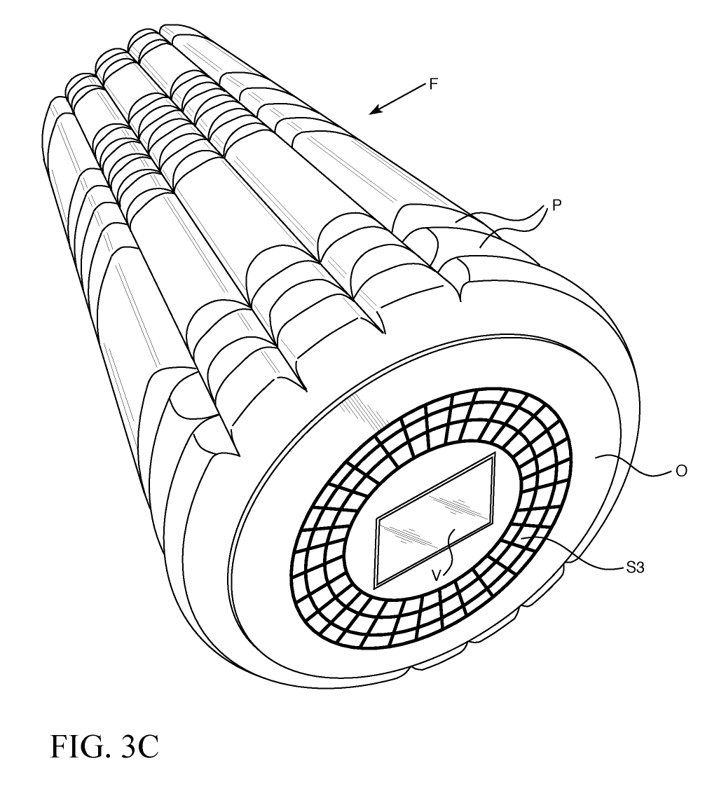

The foam roller F depicted will have at least one speaker S1 at a first end E, preferably adjacent its power button B. In FIG. 3A, the opposite end O will have a second, similarly sized and shaped speaker (also S1) for the ultimate in sound balance.

In a first alternative embodiment, FIG. 3B, the opposite end cap is provided with a smaller, round speaker S2 in an end cap that further includes a timer T, USB port aperture A and an interactive human body part display map M, the latter being touch sensitive so as to activate the timer T for the preferred duration of exercising the particular body part selected with foam roller F. Beneath timer T, there are a plurality of repetition buttons R (four are shown) for lighting up following the completion of the same exercise, multiple times, on the same body part with foam roller F.

Applicant's preferred product designs should include a timer T that correlates with the Bluetooth synchronizer during the playing of music from foam roller F. While the foam roller is being utilized for therapeutic benefits, the resettable timer can keep track of the amount of time spent for each exercise. Once that time has been completed, the Bluetooth synchronizer will disconnect or otherwise audibly notify the individual that his/her time has been completed. This will allow for proper time spent using the foam roller and can aid in therapeutic settings with proper time management and care of patients.

In the end cap depicted in FIG. 3C, there is shown a circumferential speaker S3 that surrounds a video display screen V on which may be shown specific videos for the user to watch indicating how best to exercise a particular body part/region with the foam roller F after that body part is selected, either from a drop-down menu, or a human body part display map shown on that video display screen V.

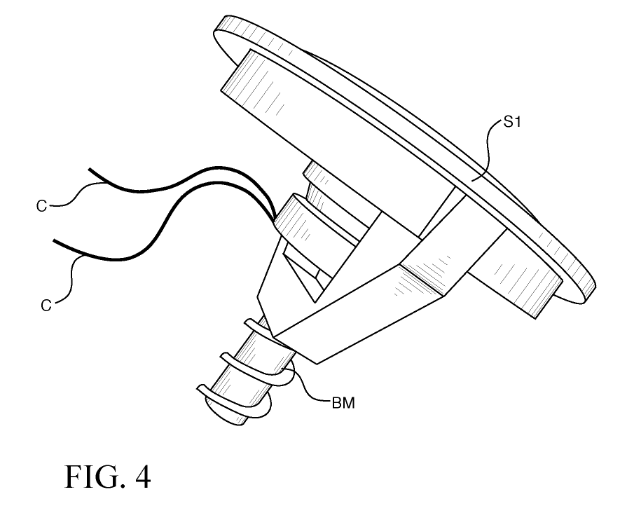

FIG. 4 shows a representative speaker S1 is side view with its wire connectors C to an internal sound board (see, FIG. 5) and its lower screw in, base mount BM for attaching to the inside frame of the foam roller F . . . at one or both ends depending on the model selected.

FIG. 5 shows one end to a typical sound board SB for use with either version of this invention, namely, a new foam roller construction, or a “retrofit” for adding to the inside cylindrical chamber of an existing foam roller model. In addition to its wire connectors C to speaker S1 in this view, sound board SB shows its central frame support FS that is a rigid, durable base capable of securely anchoring inside a cylindrical foam roller central support pipe while still withstanding the rigors of daily use (including the abuses of being periodically tossed about an exercise room, and repeatedly rolled upon for doing various body part exercises with said foam roller.

Though the aforementioned speaker and sound board components are shown in some detail in accompanying FIGS. 4 and 5, it is to be understood that Applicant's invention should NOT be limited to any one sound system, sound board, or even foam roller exterior configuration(s).

Though not shown, it is understood that the aforementioned sound board would be powered by one or more, rechargeable batteries.

It is anticipated that future improvements to Applicant's basic product design will have a smart phone app that for controlling: the timer on the foam roller, the music plated there through; or both. The true purpose of such an app would be to access a 3D model of the anatomical body so the individual can tap on the body where their soreness or pain is located. A video (or gif) describing how to properly use the foam roller for that particular body area tapped will appear. Ideally, this same phone app can include a timer with repetition buttons like that described above for FIG. 3B.

### REFERENCES

1) Curran P, Fiore R, Crisco J. A Comparison of the Pressure Exerted on Soft Tissue by 2 Myofascial Rollers. Journal Of Sport Rehabilitation [serial online]. November 2008; 17(4):432-442. Available from: SPORTDiscus with Full Text, Ipswich, Mass. Accessed Feb. 9, 2017.
2) Weller, C. M., & Baker, F. A. (2011). The role of music therapy in physical rehabilitation: A systematic literature review. Nordic Journal of Music Therapy, 20(1), 43-61. doi: 10.1080/08098131.2010.485785
3) Couture G, Karlik D, Glass S, Hatzel B. The Effect of Foam Rolling Duration on Hamstring Range of Motion. The Open Orthopedics Journal [serial online]. Oct. 2, 2015; 9:450-455. Available from: MEDLINE, Ipswich, Mass. Accessed Feb. 9, 2017.
4) Vaughan B, McLaughlin P. Immediate changes in pressure pain threshold in the iliotibial band using a myofascial (foam) roller. International Journal Of Therapy & Rehabilitation [serial online]. December 2014; 21(12):569-574. Available from: CINAHL Complete, Ipswich, Mass. Accessed Feb. 9, 2017.
5) Murray A, Jones T, Horobeanu C, Turner A, Sproule J. Sixty Seconds of Foam Rolling Does Not Affect Functional Flexibility or Change Muscle Temperature in Adolescent Athletes. International Journal Of Sports Physical Therapy [serial online]. October 2016; 11(5):765-776. Available from: SPORTDiscus with Full Text, Ipswich, Mass. Accessed Feb. 9, 2017.
6) Healey K C, Hatfield D L, Blanpied P, Dorfman L R, Riebe D (2014) The effects of myofascial release with foam rolling on performance. J Strength Cond Res 28(1): 61-8. doi: 10.1519/JSC.0b013e3182956569
7) Mohr A, Long B, Goad C. Effect of Foam Rolling and Static Stretching on Passive Hip-Flexion Range of Motion. Journal Of Sport Rehabilitation [serial online]. November 2014; 23(4):296-299. Available from: SPORTDiscus with Full Text, Ipswich, Mass. Accessed Feb. 9, 2017.
8) Alter D, O'Sullivan M, Bartel L, et al. Synchronized personalized music audio-playlists to improve adherence to physical activity among patients participating in a structured exercise program: a proof-of-principle feasibility study. Sports Medicine—Open [serial online]. 2015; 1(1):7. Available from: MEDLINE, Ipswich, Mass. Accessed Feb. 10, 2017.

Having described the presently preferred embodiments, it is to be understood that the invention may be further covered by the scope of the following provisionally filed product and method claims.

What is claimed is:

1. A foam roller for performing exercises on one or more human body parts, said foam roller having at least one speaker connected to a sound board that at least temporarily affixes to a central interior section of the foam roller, said sound board being capable of receiving and playing radio station broadcasts or music through the at least one speaker while the foam roller is being used to perform exercises.

2. The foam roller of claim 1, which is substantially cylindrically shaped.

3. The foam roller of claim 2, which has a circular shape in cross-section.

4. The foam roller of claim 1, which further includes a USB connection port for downloading a user's playlist.

5. The foam roller of claim 1, which further includes Bluetooth® connectivity.

6. The foam roller of claim 1, which further includes a resettable timer.

7. The foam roller of claim 6 wherein said timer includes a resettable indicator for a number of repetition exercises performed with the foam roller on one or more human body parts.

8. The foam roller of claim 1, which further includes a human body map adapted for touching in one or more areas to indicate a preferred minimum time to exercise with the foam roller the area or areas touched on the human body map.

9. The foam roller of claim 1, which further includes a video screen for displaying one or more preferred exercise routines using the foam roller.

10. An insert module for an existing cylindrical foam roller, said insert module including a durable sound board frame with a speaker connected at each opposed end, said sound board frame adapted for fitting inside the existing cylindrical foam roller and either: (a) receiving and playing radio station broadcasts or (b) receiving and playing downloaded music through the speakers while the existing cylindrical foam roller is being used to exercise one or more human body parts.

11. The foam roller insert module of claim 10, which further includes a USB connection port for downloading a user's playlist.

12. The foam roller insert module of claim 10, which further includes Bluetooth® connectivity.

13. The foam roller insert module of claim 10, which further includes a resettable timer.

14. The foam roller insert module of claim 13, wherein said timer includes a resettable indicator for a number of repetition exercises performed with the existing cylindrical foam roller on one or more human body parts.

15. The foam roller insert module of claim 10, which further includes a human body map adapted for touching in one or more areas to indicate a preferred minimum time to exercise with the existing cylindrical foam roller the area or areas touched on the human body map.

16. The foam roller insert module of claim 10, which further includes a video screen for displaying one or more preferred exercise routines using the foam roller.

17. An improved method for performing exercises with a cylindrical foam roller, said method comprising:
(a) providing the cylindrical foam roller with a programmable sound card having one or more speakers attached, said programmable sound card at least temporarily attached to a central recess of the cylindrical foam roller; and
(b) performing one or more exercises using the cylindrical foam roller to music played from the programmable sound card through the one or more speakers.

18. The method of claim 17 wherein the cylindrical foam roller may be programmed with customizable, downloadable music for each user.

19. The method of claim 17 wherein the cylindrical foam roller includes a repeatedly reprogrammable timer for setting exercise duration.

20. The method of claim 15 wherein the cylindrical foam roller includes a video display for showing clips on how to properly exercise different zones of the body with the cylindrical foam roller.
